(19) United States
(12) Patent Application Publication
(10) Pub. No.: US 2020/0029984 A1
(43) Pub. Date: Jan. 30, 2020

(54) Intravascular Thrombus Removal Device

(71) Applicant: SHANGHAI ACHIEVA MEDICAL SUZHOU CO., LTD, Suzhou, Jiangsu (CN)

(72) Inventors: Kai WANG, Suzhou, Jiangsu (CN); Ping YE, Suzhou, Jiangsu (CN)

(21) Appl. No.: 16/484,462

(22) PCT Filed: Apr. 20, 2017
(86) PCT No.: PCT/CN2017/000309

§ 371 Date: Aug. 8, 2019

(30) Foreign Application Priority Data
Dec. 30, 2016 (CN) 201611253290.6

(51) Int. Cl. A61B 17/221; A61B 17/12

(52) U.S. Cl. CPC A61B 17/221; A61B 2017/22094; A61B 17/12136; A61B 17/1204

(57) ABSTRACT

A retrieval device for a thrombus in a blood vessel, comprising a thrombus retrieving system and a transport system, characterized in that: the thrombus retrieving system comprises a thrombus push-back component (11) and a thrombus receiving and sealing component (12), and the transport system comprises a push component (22) and a manipulation component (21); the distal end of the push component (22) is connected to the thrombus receiving and sealing component (12), and the distal end of the manipulation component (21) is connected to the thrombus push-back component (11); the thrombus push-back component (11) can be released at the distal end of a target thrombus (50), and the thrombus receiving and sealing component (12) can be released at the proximal end of the target thrombus (50); by keeping the push component (22) fixed and drawing the manipulation component (21) back towards the proximal end direction, the thrombus push-back component (11) pushes the target thrombus (50) into the thrombus receiving and sealing component (12), and the thrombus push-back component (11) also can partially or wholly enter the thrombus receiving and sealing component (12) at the proximal end; finally, by wholly drawing back the transport system, the thrombus retrieving system enclosing the target thrombus (50) is withdrawn from the body. The retrieval device for a thrombus in a blood vessel has the advantages that the thrombus capture rate is high, a thrombus is less prone to fall off during withdrawal, the transport system runs smoothly, and a blood vessel is less likely to be harmed during thrombus retrieval.

### TECHNICAL FIELD

The present invention relates to a medical instrument for interventional therapy in the field of medical instruments, in particular to an intravascular thrombus removal device.

### BACKGROUND ART

Acute cerebral thrombosis is mainly caused by cerebrovascular thrombosis and is the most common fatal and disabling disease of the central nervous system. Cerebral thrombosis has the characteristics of high incidence rate, high disability rate, high mortality rate and high recurrence rate. According to a statistical data report in Beijing, the incidence of acute cerebral hemorrhage in Beijing has shown a significant downward trend in recent years, while the incidence of acute cerebral thrombosis has increased significantly; specifically, the proportion of acute cerebral hemorrhage in stroke has decreased from 42% to 16%, while the proportion of acute cerebral thrombosis has increased from 55.8% to 81.6%, thus cerebral thrombosis has become the most common brain disease.

Vascular recanalization is key to treating acute ischemic stroke. At present, there are mainly two treatment methods for intracranial thrombosis, namely drug thrombolysis and mechanical thrombectomy. Drug thrombolysis is performed by intravenous injection of rt-PA (tissue plasminogen activator) or urokinase to dissolve thrombi, or by intra-arterial contact thrombolysis, antiplatelet aggregation and anticoagulant drug therapy. Although thrombolytic therapy has shown to be able to greatly improve the prognosis of the nervous system, drug thrombolysis still faces some urgent problems. First of all, the thrombolytic time window is short, research by The National Institute Neurological Disorders and Stroke rt-PA Stroke Study Group (NINDS) holds that intravenous thrombolysis should be carried out within 3 hours of onset and the intra-arterial thrombolysis time window should be within 6 hours, while such a short thrombolysis time window enables only 4.5%-6.3% of patients to receive thrombolytic therapy; secondly, the vascular recanalization time, which is one of the important factors affecting clinical prognosis, of drug thrombolysis is long, at least 1-2 hours for both intravenous thrombolysis and intra-arterial thrombolysis; thirdly, thrombolytic therapy is only suitable for small thrombi, and the therapeutic effect is not ideal for large thrombi; moreover, some patients are not suitable for thrombolytic therapy.

In order to solve the above-mentioned problems of drug thrombolysis, mechanical elimination of thrombi has become a hot research topic in recent years. Mechanical thrombectomy is conducted by the following methods: thrombus suction, thrombectomy by a catcher, and thrombus breaking by laser. Thrombus suction has a good effect on small emboli, but when the emboli are large, distal emboli tend to escape, and the process is troublesome and blood vessels are hurt easily; at present, thrombectomy by a catcher is easy to operate and causes little damage to blood vessels, but oftentimes thrombi are not caught, thrombectomy is often required for many times, or suction with a guide catheter is required during catching, otherwise small fragments falling off from thrombi escape and block distal end blood vessels; and thrombus breaking by laser is difficult to operate, because if the laser energy is too low, it will be ineffective, and if the laser energy is too high, it will damage blood vessels and tend to cause various complications.

Chinese invention patent CN103417258.B discloses an intracranial vascular thrombectomy device, which comprises a thrombus retriever, a guide wire, a push-pull guide wire and an outer sheath tube, wherein the thrombus retriever is connected with the push-pull guide wire, and the installed push-pull guide wire and the thrombus retriever are pressed and held in the outer sheath tube; in an expanded position, the thrombus retriever is pushed out of the outer sheath tube; and a certain number of inner bulges are arranged on the inner wall of the thrombus retriever.

The Chinese invention patent application CN104000635.A discloses a thrombus retriever and a thrombectomy device. The thrombus retriever is of a mesh structure and defines a lumen, and is switched between a retracted position and an expanded position. The mesh structure of the, thrombus retriever is provided with a plurality of inner concave stems extending into a three-dimensional profile of the lumen, and both ends of each inner concave stem are fixed to the mesh structure.

Although the thrombus retriever with the mesh tubular structure is easy to operate, the thrombus retriever relies on the mesh per se to catch thrombi. Even if inner bulges are added or inner concave stems are connected, the situation that fragments formed after thrombi are broken during the expansion of the mesh fall off during the withdrawal of a thrombus pulling instrument cannot be avoided, and the thrombectomy effect is not reliable. At present, doctors often need to use a balloon guide catheter to overcome this problem, and a balloon is used to block blood flow to prevent the falling thrombus fragments from being flushed to distal end blood vessels by blood flow. Because of the adherence to blood vessels, if the radial force of the mesh is too large due to improper adjustment, the thrombus retriever with the mesh lumen will cause great damage to the inner wall of blood vessels.

In the utility model “Thrombus Retriever” with a patent number of ZL200620164685.4 published in China, an umbrella with two long and one short claws and an elastic memory function forms a circular thrombectomy device together with a net attached to the periphery, and by pulling out a pull rod to close the three claws, thrombi are contained in the umbrella and retrieved into an outer sleeve, thus achieving thrombus removal.

U.S. invention patent US2009/0240238.A1 discloses a thrombectomy device, which has a self-expandable snare fixed to the end of an elongated shaft and a foldable bag made of a flexible nonporous material attached thereto. The device is placed at an embolic position along an in vivo passage through the elongated shaft, and the bag is opened to wrap the thrombi.

Although the basket type thrombectomy device prevents thrombus fragments from escaping to a certain extent, the obvious defect is that the size is often too large to be used in cerebral arteries such as M1 and M2 sections of middle cerebral artery with small blood vessel diameters.

Chinese invention patent application CN201110222609.X and Chinese utility model patent CN201120281795.X disclose a thrombus suction catheter, which comprises a suction tube, wherein the suction tube comprises a tube base, the tube base is connected with a catheter body, the outer wall of the suction tube is movably sleeved with a sleeve, the sleeve comprises a Y-shaped connector, a double-cavity tube and a balloon, the Y-shaped connector is connected with the double-cavity tube, the balloon is arranged on the outer wall of a distal end of the double-cavity tube, interference wires are enclosed in the suction tube, and a distal end of each interference wire extends out of a distal end of the suction tube. The thrombectomy system quickly removes thrombi scattered in a wide range of blood vessels and treats embolism of coronary artery distal microvasculature, and thrombi with a large size and high consistency are broken up first with the interference wires before suction.

U.S. patent application US2010/0049147A1 discloses another thrombus suction catheter. U.S. patent application US2007/0161963A1 also discloses a thrombectomy suction catheter system.

The above-mentioned thrombectomy system has a good effect on small emboli, but in order to prevent blockage of the suction tube when large thrombi are removed, the thrombi need to be repeatedly mashed before suction, which is troublesome and tends to damage blood vessels.

From the above statement, it can be seen that the thrombectomy devices in the above patent documents and the existing thrombectomy technology have one or more defects. Therefore, it is necessary to further improve the prior art, and it is expected to design a better vascular thrombus retriever.

### SUMMARY OF THE INVENTION

In order to solve the above-mentioned technical problems, the object of the present invention is to provide an intravascular thrombus removal device, which has the following characteristics: firstly, the thrombus catching method with a mesh used during conventional mechanical thrombus removal is changed, and a more reasonable integral thrombus pushing and containing method is adopted, so that the thrombus catching rate of a thrombus retriever is high, the device better stabilizes thrombi, and thrombi do not fall off easily during withdrawal; secondly, the radial supporting force of the thrombus retriever needs to be moderate, or a special structural design is utilized to ensure that after the thrombi are caught, the contact area between the thrombus retriever and the inner wall of blood vessels is reduced so as to reduce the damage to vascular walls by the device as much as possible; thirdly, a delivery system is highly flexible and reaches intracranial thin distal end blood vessels; fourthly, operation is convenient, and thrombectomy is accurate and reliable; fifthly, in a clinical emergency, for example, when the thrombus retriever is entangled with a vascular stent implanted earlier in the blood vessel, a certain method is adopted to remedy the situation; sixthly, the thrombus retriever is applicable to thrombi of different lengths, and the, thrombus retriever of one specification is used to remove thrombi of different sizes; and seventhly, the thrombus retriever per se blocks the blood flow at a proximal end of the blood vessel containing thrombi, thus avoiding the impact of blood flow on thrombi during thrombectomy.

The present invention is realized through the following technical scheme:

An intravascular thrombus removal device comprises a thrombectomy system and a delivery system, and is characterized in that the thrombectomy system comprises a thrombus push-back component and a thrombus containing sealed component, the delivery system comprises a push component and a control component, a distal end of the push component is connected with the thrombus containing sealed component, and a distal end of the control component is connected with the thrombus push-back component; the thrombus push-back component is released at a distal end of a target thrombus, and the thrombus containing sealed component is released at a proximal end of the target thrombus; by keeping the push component fixed and pulling back the control component towards the proximal end, the thrombus push-back component pushes the target thrombus into the thrombus containing sealed component, the thrombus push-back component also partially or wholly enters the thrombus containing sealed component at the proximal end, and finally the thrombectomy system in which the target thrombus is wrapped is withdrawn from the body by integrally pulling back the delivery system.

The thrombus push-back component is released at the distal end of the target thrombus, and the thrombus containing sealed component is released at the proximal end of the target thrombus; by keeping the push component fixed and pulling back the control component towards the proximal end, the thrombus push-back component connected to the control component pushes the whole thrombus to move towards the proximal end along a blood vessel, and the whole target thrombus is integrally pushed into the thrombus containing sealed component located at the proximal end. At this point, the thrombus push-back component also enters the thrombus containing sealed component integrally or partially, the thrombus push-back component serves as a sealed cover of the thrombus containing sealed component, and finally the thrombus is completely in a sealed cage formed by the thrombus push-back component and the thrombus containing sealed component. Then in the process of withdrawing the device and the thrombus from the body, the thrombus will be kept in the, cage formed by the thrombus push-back component and the thrombus containing sealed component in the whole process, thus effectively preventing the thrombus from being completely destroyed to form fragments during withdrawing, and even if thrombus fragments are formed, the fragments will be kept in the cage, so as to prevent the thrombus fragments from moving to blood vessels at the distal end, which blocks distal small blood vessels to cause massive cerebral infarction.

In the context of present invention, the terms “distal end” and “proximal end” should be understood as viewed from the perspective of an attending physician. The distal end is thus the side away from the attending physician, while the proximal end represents the side toward the attending physician. If the phrase “axial” is used in this document, “axial” is interpreted to indicate the direction in which the device of the present invention is advanced, that is, the longitudinal axis of the device also coincides with the longitudinal axis of a blood vessel along which the device moves forward. “Sealed” in the thrombus containing sealed component does not mean that the structure of the thrombus containing sealed component must be a completely sealed object, and the structure thereof is also similar to that of a cage. The purpose of “sealed” is only to express one characteristic of the device, namely, the thrombus containing sealed component forms a space together with the thrombus push-back component, the thrombus is contained in the space, and the thrombus does not escape from the space easily when the thrombus is withdrawn from the body along with the thrombectomy system.

In one example of the present invention, the push component is an elongated delivery tube, and the control component is an elongated traction wire which penetrates into an inner cavity of the delivery tube and moves freely and axially within the inner cavity. The delivery system consisting of the delivery tube and the traction wire delivers the thrombectomy system to the position of the thrombus along a microcatheter. Before thrombectomy, the doctor adjusts the distance between the thrombus push-back component and the thrombus containing sealed component by adjusting the delivery tube and the traction wire outside the body, thus realizing the function of removing thrombi of different lengths by only using the device of the same specification.

To further illustrate the above example, a most proximal end of the thrombus push-back component is fixedly connected with a most distal end of the traction wire, and a most proximal end of the thrombus containing sealed component is fixedly connected with a most distal end of the delivery tube.

To further explain the above example, the most distal end of the traction wire passes through the most proximal end of the thrombus push-back component to be fixedly connected with the most distal end of the thrombus push-back component, and the most proximal end of the thrombus containing sealed component is fixedly connected with the most distal end of the delivery tube.

To further illustrate the above example, the delivery tube is an elongated metal tube, and the distal end thereof is cut to half within a certain length or carved into hollow texture. By reducing the material at the distal end of the delivery tube, the distal end portion of the delivery tube is softer, and the delivery system more easily reaches tortuous blood vessels at the distal end of the brain.

To further illustrate the above example, the delivery tube is an elongated tube formed by splicing of different materials, and the joints thereof are connected through clamping, bonding or welding. By means of clamping, bonding or welding, different positions of the same delivery tube have different hardnesses, for example, the portion, at a proximal end, of is made harder so as to provide stronger pushing, while the portion, at the distal end, of the delivery tube is made softer so as, to ensure the passing ability in the tortuous blood vessels.

In another example of the present invention, the thrombus push-back component and the thrombus containing sealed component are metal stents carved from a shape memory alloy tube and subjected to post-setting treatment, or dense mesh fabrics braided from metal wires and subjected to post-setting treatment. The metal stent made of shape memory alloy has sufficient radial supporting force to ensure good adherence, so that when the metal stent is used as the thrombus containing sealed component, the periphery of a distal end opening thereof stick to the inner wall of the blood, vessel to prevent the pushed-back thrombus from entering a gap between the metal stent and the blood vessel wall, and the metal stent comprises interconnected ribs to form a plurality of grid cells, so that when the metal stent is used as the thrombus push-back component, the unobstructed blood flow flowing to the distal end blood vessel is ensured while the blood vessel is pushed back by a surface formed by the grid cells. The dense mesh fabric braided from metal wires has a softer supporting force, so that the blood vessel does not get hurt easily when the dense mesh fabric is used as the thrombus containing sealed component; compared with the grid cells of the metal stent, the mesh area of the dense mesh fabric is smaller, so that when used as, the thrombus push-back component, the dense mesh fabric pushes small or softer thrombi and thrombus fragments more easily. The thrombus push-back component and the thrombus containing sealed component are selected and matched according to the actual condition of a patient, for example, the thrombus push-back component adopts a dense mesh fabric, and the thrombus containing sealed component adopts a metal stent.

In another example of the present invention, the proximal and distal end portions of the thrombus push-back component are both closed up and sealed using a ring or spring coil, so as to form a lemon-like shape. The lemon-shaped thrombus push-back component makes a smaller contact surface between the component and the inner wall of the blood vessel, thus reducing the probability of damage to the blood vessel by the device. While a surface formed by the closing of the proximal end of the lemon-shaped thrombus push-back component pushes back the thrombus, a surface formed by the closing of the distal end blocks the broken thrombus leaking from the surface formed by the closing of the proximal end, thus providing more powerful protection for distal end blood vessels.

In another example of the present invention, only the proximal end portion of the thrombus push-back component is closed up and sealed using a ring or spring coil, so as to form a bag shape. The bag-shaped thrombus push-back component has good adherence. When the thrombus is soft, the good adherence thereof prevents the thrombus from passing through the outer circumference of the thrombus push-back component and escaping to distal end blood vessels.

In another example of the present invention, only the proximal end portion of the thrombus containing sealed component is closed up and sealed using a ring or spring coil, so as to form a bag shape. The bag-shaped thrombus containing sealed component has good adherence, so that the distal end opening thereof is larger, which contains the thrombus more easily.

In another example of the present invention, the proximal end of the thrombus containing sealed component comprises a blood flow blocking element, and the blood flow blocking element is a membrane adhering to the outer surface of the proximal end of the thrombus containing sealed component. When the thrombus containing sealed component is completely expanded at the blood vessel and is tightly attached to the blood vessel wall, the membrane at the proximal end portion of the thrombus containing sealed component will block blood flow, thus preventing blood from impacting the thrombus and preventing thrombus fragments from falling off and escaping to distal end blood vessels during thrombectomy.

In another example of the present invention, the proximal end of the thrombus containing sealed component comprises a blood flow blocking element, the blood flow blocking element is a balloon installed at the proximal end of the thrombus containing sealed component, and the push component is a double-cavity tube or an inner and outer sleeve tube. This example relies on the balloon of the device per se to block the blood flow at the proximal end of the blood vessel.

In another example of the present invention, the thrombus containing sealed component comprises an outer diameter tightening element, the outer diameter tightening element is structured as at least one tightening wire wound in a spiral manner on the circumferential outer surface of the thrombus containing sealed component, a distal end of the tightening wire is fixedly connected with the most distal end of the thrombus containing sealed component, and the outer diameter of the thrombus containing sealed component is reduced under the action of the tightening wire when the proximal end of the tightening wire is pulled back.

To further explain the above example, the delivery system comprises a tightening traction wire, and the push component is a double-cavity tube, or a single-cavity tube with one side, opening at each of the side surfaces of two side nozzles at the most proximal end and the most distal end. The tightening traction wire passes through the inner cavity of the push component, and a most distal end of the tightening traction wire is fixedly connected with a most proximal end of the tightening wire on the thrombus containing sealed component. After a clinician operates the thrombus push-back component to push the thrombus into the thrombus containing sealed component, the clinician pulls back the tightening traction wire, resulting in a reduction in the length of the tightening wire spirally wound at the thrombus containing sealed component and a reduction in the diameter of the thrombus containing sealed component. At this point, the outer surface of the thrombus containing sealed component will no longer completely abut against the inner wall of the blood vessel, thus reducing the damage of the blood vessel wall due to the reduction of the contact area between the device and the blood vessel in the subsequent withdrawal process of the device.

In another example of the present invention, the region between the distal end of the push component and the proximal end of the thrombus containing sealed component forms an electrically releasable region, and the region between the distal end of the control component and the proximal end of the thrombus push-back component forms an electrically releasable region. When the device gets stuck in the body during the thrombectomy process, for example, a stent is used in carotid artery blood vessel or intracranial blood vessel, if the mesh on the thrombus retriever is connected with the mesh of the stent, forcible withdrawal of the thrombus retriever is bound to seriously damage the blood vessel. At this point, the thrombus push-back component or thrombus containing sealed component of the thrombus retriever is left in the blood vessel by means of electric release, and then the rest of the thrombus retriever is withdrawn from the body, which is convenient for doctors to carry out the following rescue. In this example, the principle of electrochemical corrosion is used to realize release, for example, the release principle of an electro-release spring coil for embolization of intracranial aneurysm is common in the market at present.

In another example of the present invention, the delivery system comprises an operating handle, wherein the operating handle comprises a holding handle, a push-pull button and a locking valve; a front end of the holding handle is provided with an opening; when the locking valve is opened, the delivery system extends into the opening; and when the locking valve is closed, the, push component is clamped by the locking valve, the control component is clamped by the push-pull button, and relative movement between the push component and the control component is controlled by operating the push-pull button. By using the operating handle, doctors more easily control the axial relative positions of the thrombus push-back component and the thrombus containing sealed component in the operation process.

In another example of the present invention, a polymer membrane is attached to the surface of the thrombus push-back component or the thrombus containing sealed component. Adding the membrane enhances the thrombus pushing capability of the thrombus push-back component, which is more effective for soft thrombi and thrombus fragments. The membrane attached to the surface of the thrombus containing sealed component more effectively prevents thrombi from escaping during withdrawal, and the preferred polymer membrane is polyurethane or PTFE.

In another example of the present invention, the thrombus push-back component and thrombus containing sealed component are made of super-elastic shape memory alloy, so as to be able to retract into an outer catheter in a folded mode under the action of external force; when the restriction of the outer catheter is removed, a thrombectomy system is unfolded back to the, original shape thereof; the thrombectomy system is moved back and forth in an inner cavity of the outer catheter by controlling the delivery system. The alloy material is nitinol alloy in particular. Due to the superelasticity of the shape memory alloy, it is ensured that the thrombus push-back component and the thrombus containing sealed component are compressed and folded into a very small size, and are adaptable to blood vessels and catheters with various diameters, such as microcatheters, guide sheaths and guide catheters.

In another example of the present invention, both the most proximal end and the most distal end of the thrombus push-back component or the thrombus containing sealed component contain X-ray impermeable markers, and the X-ray impermeable marker is a developing ring or a developing spring coil. Clinicians monitor the locations of the X-ray impermeable markers through vascular imaging equipment such as DSA (Digital Subtraction Angiography) to further understand the status of the device and the progress of treatment.

In another example of the present invention, the surface of the thrombus push-back component or the thrombus containing sealed component is provided with an X-ray impermeable coating. The X-ray impermeable coating makes the whole thrombectomy system completely displayed under the vascular imaging equipment, which is convenient for doctors to monitor and operate.

To further explain the above example, the X-ray impermeable markers are made of platinum iridium alloy or platinum tungsten alloy or platinum iridium tungsten alloy.

In another example of the present invention, the surface of the push component or the control component is coated with a lubricating coating, and the coating is a PTFE coating or hydrophilic coating. The lubricating coating reduces the resistance in the pushing process and increases the handling performance of the device.

In another example of the present invention, the delivery system comprises a guide sheath made of the polymer material polytetrafluoroethylene. The thrombectomy system retracts into the guide sheath, and the inner diameter of the guide sheath is equal to the inner diameter of the microcatheter, so that the whole device is conveniently sent into the microcatheter.

Compared with the prior art, the present invention has the following advantages: firstly, the thrombus catching method with a mesh used during conventional mechanical thrombus removal is changed, and a more reasonable integral thrombus pushing and containing method is adopted, so that the thrombus catching rate of the thrombus retriever is high, the device better stabilizes thrombi, and thrombi do not fall off easily during withdrawal;

secondly, the thrombus retriever utilizes a tightening method using the tightening wire to ensure that the external diameter of the thrombectomy system is reduced after thrombus catching, and the contact area between the thrombus retriever and the inner wall of blood vessels is reduced so as to reduce the damage to blood vessels by the thrombus retriever;

thirdly, the delivery system is highly flexible and reaches, for example, intracranial thin distal end blood vessels;

fourthly, the operating handle is used to make thrombectomy convenient to achieve, accurate and reliable;

fifthly, the function of electric release provides a solution for clinicians in an emergency;

sixthly, clinicians adjust the distance between the thrombus push-back component and the thrombus containing sealed component before operation to realize that the device of the same specification is applied to thrombi of different lengths, so manufacturers only need to design and manufacture the thrombus retriever of one specification, and the stock pressure of hospitals is also reduced; and

seventhly, the thrombus retriever per se blocks the blood flow at the proximal end of the blood vessel containing thrombi, thus avoiding the impact of blood flow on thrombi during thrombectomy and preventing thrombi from escaping to the distal end.

### BRIEF DESCRIPTION OF THE DRAWINGS

Examples of the present invention will be explained by the following drawings, in which:

FIG. 1 is a schematic structural diagram of an example of an intravascular thrombus removal device of the present invention;

FIG. 2 is a cross-sectional view of a proximal end portion of a thrombus containing sealed component in the example shown in FIG. 1;

FIGS. 3-7 are schematic views of the process of removing an intravascular target thrombus in the example shown in FIG. 1;

FIG. 8 is a schematic structural diagram of another example of an intravascular thrombus removal device of the present invention, wherein a most distal end of a control component passes through a most proximal end of a thrombus push-back component to be fixedly connected with a most distal end of the thrombus push-back component;

FIG. 9 is a schematic structural diagram of another example of an intravascular thrombus removal device of the present invention, wherein a proximal end of a thrombus containing sealed component comprises a blood flow blocking element;

FIG. 10 is a schematic structural diagram of another example of an intravascular thrombus removal device of the present invention, wherein a thrombus containing sealed component comprises an outer diameter tightening element;

FIG. 11 is a cross-sectional view of a proximal end portion of the thrombus containing sealed component in the example shown in FIG. 10;

FIG. 12 is a cross-sectional view of a proximal end portion of a push component in the example shown in FIG. 10;

FIG. 13 is a schematic view showing the outer diameter tightening element in the example shown in FIG. 10 reducing the outer diameter of the thrombus containing sealed component;

FIG. 14 is a schematic structural diagram of another example of an intravascular thrombus removal device of the present invention, wherein a thrombus push-back component is a dense mesh fabric braided from metal wires and subjected to post-setting treatment;

FIG. 15 is a schematic structural diagram of an example of an operating handle included in a delivery system of the present invention;

FIG. 16 is a schematic structural diagram of an example of a thrombus push-back component of the present invention, wherein only a proximal end of the thrombus push-back component is closed up, so as to form a bag shape;

FIG. 17 is a schematic structural diagram of an example of a thrombus push-back component of the present invention, wherein the outer surface of a proximal end of the thrombus push-back component is coated with a polymer membrane; and

FIG. 18 is a schematic structural diagram of an example of a thrombus containing sealed component of the present invention, wherein the thrombus containing sealed component is a dense mesh fabric braided from metal wires and subjected to post-setting treatment.

### DETAILED DESCRIPTION OF THE INVENTION

According to the principles of the present invention, with reference to the above drawings, several examples of a thrombus retriever are disclosed herein. However, the examples disclosed herein are only some examples of the present invention. The details disclosed only serve as the basis for the application of the claims and impart how to properly apply the present invention to those with relevant technical background.

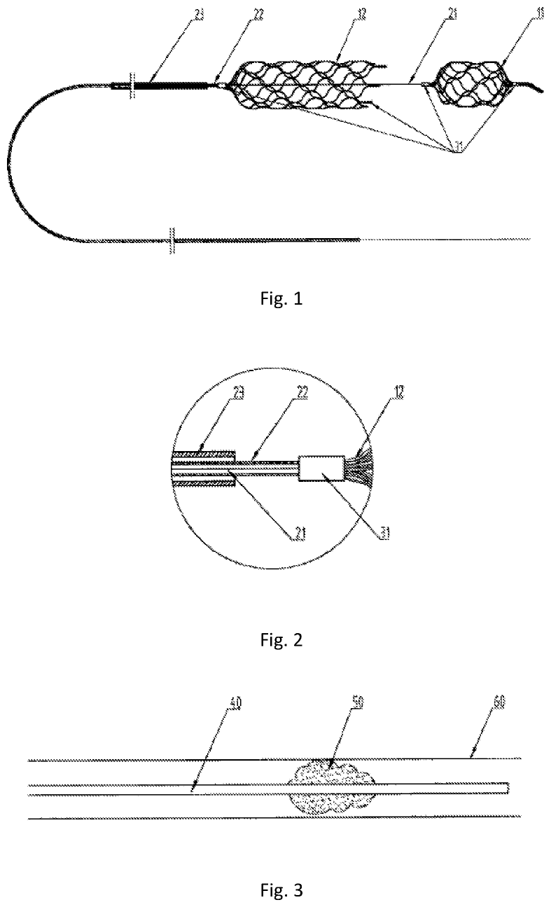

FIG. 1 shows an example of an intravascular thrombus removal device of the present invention. A thrombectomy system includes a thrombus push-back component (11) and a thrombus containing sealed component (12), both of which are metal stents made of laser engraving nickel-titanium alloy tubes and subjected to post-setting treatment. A proximal end portion and a distal end portion of the thrombus push-back component (11) are closed up to form a lemon-like shape, and only a proximal end portion of the thrombus containing sealed component (12) is closed up to form a bag shape. The most distal ends of the thrombus push-back component (11) and the thrombus containing sealed component (12) are both provided with X-ray impermeable markers (31) with a developing spring coil structure, and the most proximal ends of the thrombus push-back component (11) and the thrombus containing sealed component (12) are, both provided with X-ray impermeable markers (31) with a developing ring structure. A delivery system includes a control component (21) in the form of an elongated traction wire and a push component (22) in the form of a delivery tube, wherein the control component (21) passes through an inner cavity of the push component (22), the length of the control component (21) is preferably 210 cm, and the length of the push component (22) is preferably 200 cm. A most proximal end of the thrombus push-back component (11) is fixedly connected with a most distal end of the control component (21) in the form of a traction wire, and a most proximal end of the thrombus containing sealed component (12) is fixedly connected with a most distal end of the push component (22) in the form of a delivery tube. The delivery system further includes a guide sheath (23), wherein the guide sheath (23) is made of the polymer material polytetrafluoroethylene, the thrombectomy system retracts into the guide sheath (23) in a folded mode, the inner diameter of the guide sheath (23) is equal to the inner diameter of a microcatheter (40), and the length is preferably 65 mm.

FIG. 2 shows a cross-sectional view of the proximal end portion of the thrombus containing sealed component (12) in the example, shown in FIG. 1, showing the control component (21) in an enlarged manner which freely moves axially within the inner cavity of the push component (22).

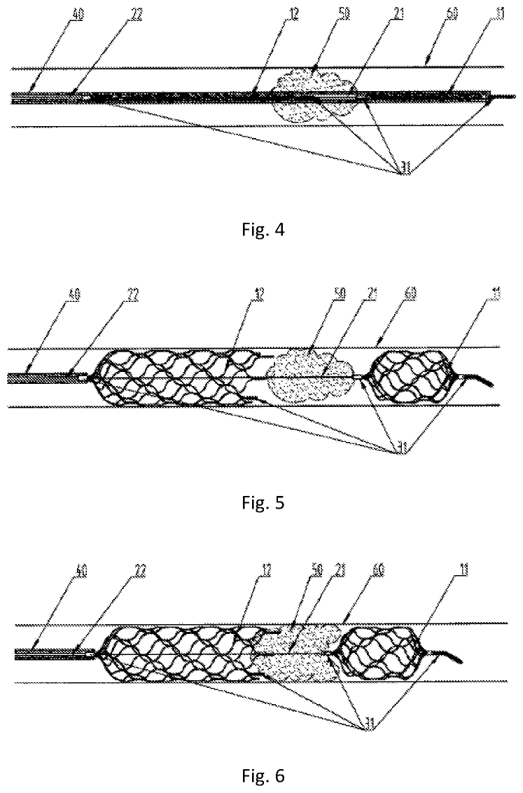

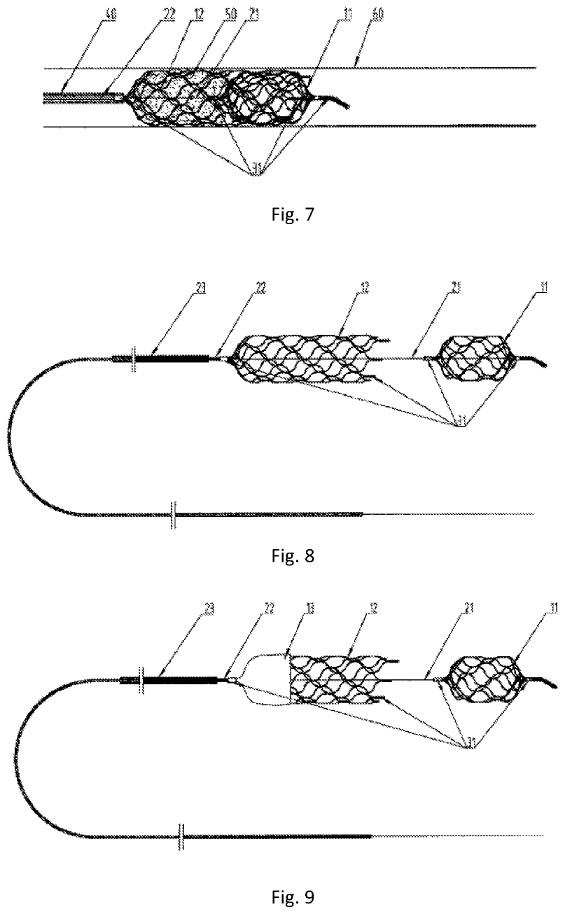

The following is an explanation of a clinician using the example shown in FIG. 1: before using the example device, the clinician uses vascular imaging equipment, such as DSA (Digital Subtraction Angiography), to measure the position and size of a target thrombus (50) in a blood vessel (60), and after confirming the rough length of the target thrombus (50), the clinician holds the push component (22) and adjusts the control component (21) by pushing and pulling, so as to adjust the distance between the thrombus push-back component (11) and the thrombus containing sealed component (12), wherein the distance is slightly larger than the length of the target thrombus (50); and then the most proximal end portions of the control component (21) and the push component (22) are fixed, so that the relative positions of the control component (21) and the push component (22) are not changed anymore, thus making the distance between the thrombus push-back component (11) and the thrombus containing sealed component (12) stay the same, and then the thrombus push-back component (11) and the thrombus containing sealed component (12) are enabled to retract in the guide sheath (23) in a folded mode. Before the clinician delivers the example device to the, microcatheter (40), a distal end of the microcatheter (40) must be delivered to a position which exceeds the target thrombus (50) by about the length of one thrombus push-back component (11), i.e., the position shown in FIG. 3. The example shown in FIG. 1 is then fed into the microcatheter (40) and the entire device is gradually pushed under the guiding of the guide sheath (23). When the x-ray impermeable marker (31) at the distal end of the thrombus push-back component (11) reaches the most distal end of the microcatheter (40), i.e., the position shown in FIG. 4, the whole of the example shown in FIG. 1 is kept motionless with respect to the human body and the microcatheter (40) is withdrawn. With the withdrawal of the microcatheter (40), the thrombus push-back component (11) and the thrombus containing sealed component (12) will be expanded at a distal end and a proximal end of the target thrombus (50) respectively, at positions as shown in FIG. 5, and the most distal end of the microcatheter (40) is withdrawn continuously to the position of the x-ray impermeable marker (31) at the proximal end of the thrombus containing sealed component (12). Over 3 minutes later, the thrombus push-back component (11) and the thrombus containing sealed component (12) are completely expanded, then the clinician holds the push component (22) and pulls back the control component (21), as shown in FIG. 6, and at this point, the thrombus push-back component (11) will also push the target thrombus (50). The clinician continues to pull back the control component (21) until the target thrombus (50) is completely pushed into the thrombus containing sealed component (12), and the thrombus push-back component (11) also partially or completely enters the thrombus containing sealed component (12), i.e., at the position shown in FIG. 7. The thrombus push-back component (11) plays a sealing role at this point, to prevent the thrombus from escaping in the following withdrawal process. When the clinician judges that the target thrombus (50) is well caught, the whole example shown in FIG. 1 is quickly withdrawn, and when the thrombus containing sealed component (12) containing the target thrombus (50) is, withdrawn to an entrance of a guide catheter, the inner diameter of the guide catheter is larger than the inner diameter of the microcatheter (40), but smaller than the inner diameter of the blood vessel (60), which will inevitably lead to deformation of the components of the whole thrombectomy system, and the target thrombus (50) is bound to be compressed and deformed along with the whole thrombectomy system; however, the target thrombus (50) will not escape from the thrombectomy system because the target thrombus (50) is already locked in a cage formed by the thrombus push-back component (11) and the thrombus containing sealed component (12). The clinician continues to withdraw the example device, and finally the thrombectomy system will withdraw the example device containing the target thrombus (50) out of the body to complete the whole thrombectomy process.

FIG. 8 is another example of an intravascular thrombus removal device of the present invention, which is different from the example shown in FIG. 1 in that the most distal end of the control component (21) in the form of a traction wire passes through the most proximal end of the thrombus push-back component (11) to be fixedly connected with the most distal end of the thrombus push-back component (11), and the most proximal end of the thrombus containing sealed component (12) is fixedly connected with the most distal end of the push component (22) in the form of a delivery tube.

FIG. 9 shows an example of another intravascular thrombus removal device of the present invention, which is different from the example shown in FIG. 1 in that a blood flow blocking element (13) is included at the proximal end of the thrombus containing sealed component (12), the blood flow blocking element (13) is structurally a membrane coated on the outer surface of the proximal end of the thrombus containing sealed component (12), and the material is preferably polyurethane. In the thrombectomy process, when the most distal end of the microcatheter (40) is withdrawn to the position of the x-ray impermeable marker (31) at the proximal end of the thrombus containing sealed component (12), the thrombus containing sealed component (12) will be fully expanded and will be tightly attached to the blood vessel wall, and meanwhile the blood flow blocking element (13) will also be fully expanded and block the blood flow at this position, thus preventing blood from impacting the thrombus and preventing thrombus fragments from falling off and escaping to distal end blood vessels during thrombectomy.

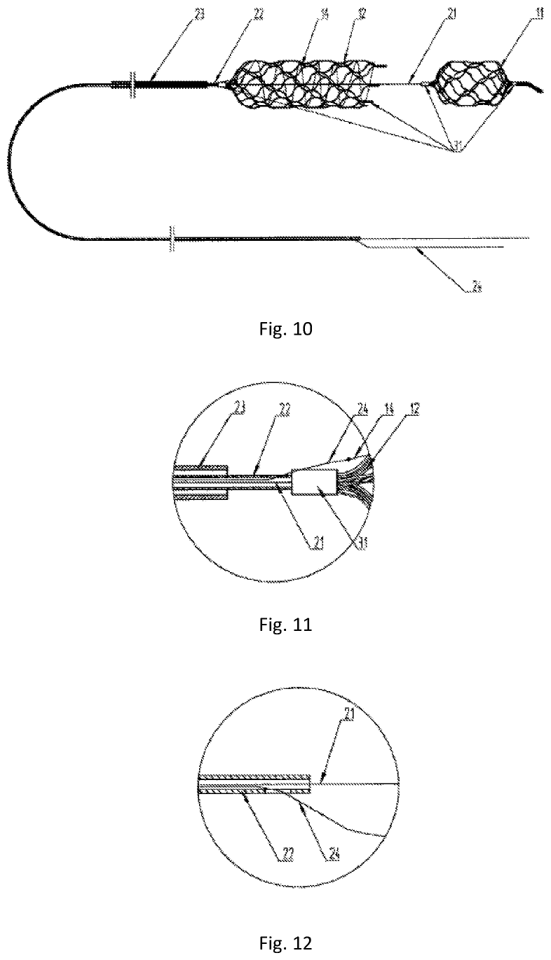

FIG. 10 shows another example of an intravascular thrombus removal device of the present invention, which is different from the example shown in FIG. 1 in that the thrombus containing sealed component (12) includes an outer diameter tightening element (14), the outer diameter tightening element (14) is structurally at least one tightening wire wound in a spiral manner on the circumferential outer surface of the thrombus containing sealed component (12), and a distal end of the tightening wire is fixedly connected with the most distal end of the thrombus containing sealed component (12). The delivery system includes a tightening traction wire (24), and the push component (22) in this example is structurally a single-cavity tube with one side opening at each of the side surfaces of two side nozzles at the most proximal end and the most distal end. The tightening traction wire passes through the inner cavity of the push component (22), and a most distal end of the tightening traction wire (24) is fixedly connected with a most proximal end of the outer diameter tightening element (14) on the thrombus containing sealed component (12). FIG. 11 shows a cross-sectional view of the proximal end portion of the thrombus containing sealed component (12) in the example shown in FIG. 10, and FIG. 12 is a cross-sectional view of the proximal end portion of the push component (22) in the example shown in FIG. 10.

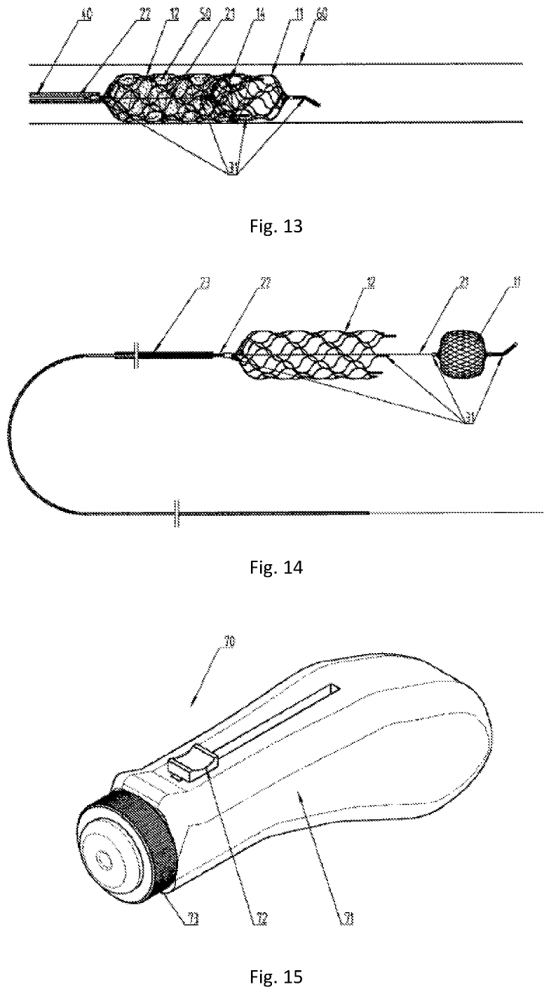

After the clinician operates the thrombus push-back component (11) to push the target thrombus (50) into the thrombus containing sealed component (12), the clinician pulls back the tightening traction wire (24), resulting in a reduction in the length of the outer diameter tightening element (14) in the form of a tightening wire spirally wound at the thrombus containing sealed component (12) and a reduction in the diameter of the thrombus containing sealed component (12). At this point, the outer surface of the thrombus containing sealed component (12) will no longer completely abut against the inner wall of the blood vessel (60), as shown in FIG. 13, thus reducing the damage of the blood vessel wall due to the reduction of the contact area between the device, and the blood vessel (60) in the subsequent withdrawal process of the device.

FIG. 14 is another example of an intravascular thrombus removal device of the present invention, which is different from the example shown in FIG. 1 in that the thrombus push-back component (11) is a dense mesh fabric braided from metal wires and subjected to post-setting treatment, and the proximal end portion and the distal end portion of the thrombus push-back component (11) are closed up to form a lemon-like shape. The mesh area of the dense mesh fabric is smaller, so that the dense mesh fabric pushes small or softer thrombi and thrombus fragments more easily.

FIG. 15 shows an example of an operating handle (70) included in a delivery system of the present invention. The operating handle (70) includes a holding handle (71), a push-pull button (72) and a locking valve (73); a front end of the holding handle (71) is provided with an opening; when the locking valve (73) is opened, the delivery system extends into the opening; and when the locking valve (73) is closed, the push component (22) is clamped by the locking valve (73), the control component (21) is clamped by the push-pull button (72), and relative movement between the push component (22) and the control component (21) is controlled by operating the push-pull button (72). By using the operating handle (70), doctors more easily control the axial relative positions of the thrombus push-back component (11) and the thrombus containing sealed component (12) in the operation process.

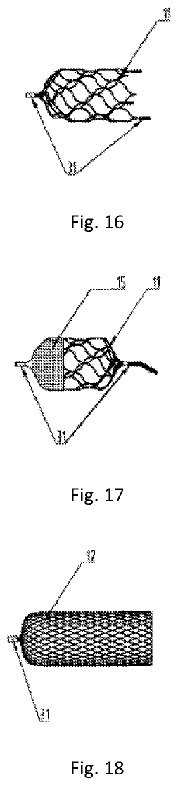

FIG. 16 shows an example of the thrombus push-back component (11) of the present invention. The thrombus push-back component (11) is a metal stent made of a laser engraving nickel-titanium alloy tube and subjected to post-setting treatment, and only the proximal end of the thrombus push-back component (11) is closed up to form a bag shape.

FIG. 17 shows an example of the thrombus push-back component (11) of the present invention. The thrombus push-back component (11) is a metal stent made of a laser engraving nickel-titanium alloy tube and subjected to post-setting treatment, and the proximal end portion and the distal end portion of the thrombus push-back component (11) are closed up to form a lemon-like shape. The outer surface of the proximal end of the thrombus push-back component (11) is coated with a polymer membrane (15), micropores are formed in the polymer membrane (15), and the material is preferably polyurethane. The additional polymer membrane (15) enhances the thrombus pushing capability of the thrombus push-back component (11), which is more, effective for soft thrombi and thrombus fragments. Besides, the micropores reduce the influence on blood flow during thrombus pushing.

FIG. 18 shows an example of the thrombus containing sealed component (12) of the present invention. The thrombus containing sealed component (12) is a dense mesh fabric braided from metal wires and subjected to post-setting treatment, and only the proximal end of the thrombus containing sealed component (12) is closed up to form a bag-like shape.

1. An intravascular thrombus removal device, comprising a thrombectomy system and a delivery system, and characterized in that the thrombectomy system comprises a thrombus push-back component and a thrombus containing sealed component, the delivery system comprises a push component and a control component, a distal end of the push component is connected with the thrombus containing sealed component, and a distal end of the control component is connected with the thrombus push-back component; the thrombus push-back component is released at a distal end of a target thrombus, and the thrombus containing sealed component is released at a proximal end of the target thrombus; and by keeping the push component fixed and pulling back the control component towards the proximal end, the thrombus push-back component pushes the target thrombus into the thrombus containing sealed component, the thrombus push-back component also partially or wholly enters the thrombus containing sealed component at the proximal end, and finally the thrombectomy system in which the target thrombus is wrapped is withdrawn from the body by integrally pulling back the delivery system.

2. The intravascular thrombus removal device according to claim 1, characterized in that the push component is structurally an elongated delivery tube, and the control component is structurally an elongated traction wire penetrating into an inner cavity of the delivery tube and moving freely and axially within the inner cavity.

3. The intravascular thrombus removal device according to claim 2, characterized in that a most proximal end of the thrombus push-back component is fixedly connected with a most distal end of the traction wire, and a most proximal end of the thrombus containing sealed component is fixedly connected with a most distal end of the delivery tube.

4. The intravascular thrombus removal device according to claim 2, characterized in that the most distal end of the traction wire passes through the most proximal end of the thrombus push-back component to be fixedly connected with the most distal end of the thrombus push-back component, and the most proximal end of the thrombus containing sealed component is fixedly connected with the most distal end of the delivery tube.

5. The intravascular thrombus removal device according to claim 1, characterized in that the thrombus push-back component and the thrombus containing sealed component are structurally metal stents carved from a shape memory alloy tube and subjected to post-setting treatment, or dense mesh fabrics braided from metal wires and subjected to post-setting treatment.

6. The intravascular thrombus removal device according to claim 1, characterized in that the proximal and distal end portions of the thrombus push-back component are both closed up and sealed using axing or spring coil, so as to form a lemon-like shape.

7. The intravascular thrombus removal device according to claim 1, characterized in that only the proximal end portion of the thrombus push-back component is closed up and sealed using a ring or spring coil, so as to form a bag shape.

8. The intravascular thrombus removal device according to claim 1, characterized in that only the proximal end portion of the thrombus containing sealed component is closed up and sealed using a ring or spring coil, so as to form a bag shape.

9. The intravascular thrombus removal device according to claim 1, characterized in that the proximal end of the thrombus containing sealed component comprises a blood flow blocking element, and the blood flow blocking element is a membrane adhering to the outer surface of the proximal end of the thrombus containing sealed component.

10. The intravascular thrombus removal device according to claim 1, characterized in that the proximal end of the thrombus containing sealed component comprises a blood flow blocking element, the blood flow blocking element is a balloon installed at the proximal end of the thrombus containing sealed component, and the push component is structurally a double-cavity tube or an inner and outer sleeve tube.

11. The intravascular thrombus removal device according to claim 1, characterized in that the thrombus containing sealed component comprises an outer diameter tightening element, the outer diameter tightening element is structurally at least one tightening wire wound in a spiral manner on the circumferential outer surface of the thrombus containing sealed component, a distal end of the tightening wire is fixedly connected with the most distal end of the thrombus containing sealed component, and the outer diameter of the thrombus containing sealed component is reduced under the action of the tightening wire when a proximal end of the tightening wire is pulled back.

12. The intravascular thrombus removal device according to claim 11, characterized in that the delivery system comprises a tightening traction wire, and the push component is a double-cavity tube, or a single-cavity tube with one side opening at each of the side surfaces of two side nozzles at the most proximal end and the most distal end; and the tightening traction wire passes through the inner cavity of the push component, and a most distal end of the tightening traction wire is fixedly connected with a most proximal end of the tightening wire on the thrombus containing sealed component.

13. The intravascular thrombus removal device according to claim 1, characterized in that the region between the distal end of the push component and the proximal end of the thrombus containing sealed component forms an electrically releasable region, and the region between the distal end of the control component and the proximal end of the thrombus push-back component forms an electrically releasable region.

14. The intravascular thrombus removal device according to claim 1, characterized in that the delivery system comprises an operating handle, the operating handle comprises a holding handle, a push-pull button and a locking valve, and a front end of the holding handle is provided with an opening; when the locking valve is opened, the delivery system extends into the opening; and when the locking valve is closed, the push component is clamped by the locking valve, the control component is clamped by the push-pull button, and relative movement between the push component and the control component is controlled by operating the push-pull button.

15. The intravascular thrombus removal device according to claim 1, characterized in that a polymer membrane is attached to the surface of the thrombus push-back component or the thrombus containing sealed component.
